(19) United States
(12) Patent Application Publication
(10) Pub. No.: US 2023/0022929 A1
(43) Pub. Date: Jan. 26, 2023

(54) COMPUTER ASSISTED SURGERY SYSTEM, SURGICAL CONTROL APPARATUS AND SURGICAL CONTROL METHOD

(71) Applicant: Sony Group Corporation, Tokyo (JP)

(72) Inventors: Christopher WRIGHT, London (GB); Bernadette ELLIOTT-BOWMAN, London (GB); Taro AZUMA, Tokyo (JP); Yohei KURODA, Tokyo (JP)

(73) Assignee: Sony Group Corporation, Tokyo (JP)

(21) Appl. No.: 17/785,911

(22) PCT Filed: Dec. 11, 2020
(86) PCT No.: PCT/JP2020/046354

§ 371 Date: Jun. 16, 2022

(30) Foreign Application Priority Data
Dec. 23, 2019 (EP) 19219485.0

(51) Int. Cl. A61B 34/10; A61B 34/20; A61B 34/37; G06T 7/00; G06V 10/25; G06V 10/82

(52) U.S. Cl. CPC A61B 34/10; A61B 34/20; A61B 34/37; G06T 7/0014; G06V 10/25; G06V 10/82; A61B 2034/2065; G06T 2207/20084; G06T 2207/20132; G06T 2207/30004

(57) ABSTRACT

A computer assisted surgery system comprising: a computerised surgical apparatus; and a control apparatus; wherein the control apparatus comprises circuitry configured to: receive information indicating a first region of a surgical scene from which information is obtained by the computerised surgical apparatus to make a decision; receive information indicating a second region of the surgical scene from which information is obtained by a medical professional to make a decision; determine if there is a discrepancy between the first and second regions of the surgical scene; and if there is a discrepancy between the first and second regions of the surgical scene: perform a predetermined process based on the discrepancy.

### FIELD

The present disclosure relates to a computer assisted surgery system, surgical control apparatus and surgical control method.

### BACKGROUND

The “background” description provided herein is for the purpose of generally presenting the context of the disclosure. Work of the presently named inventors, to the extent it is described in the background section, as well as aspects of the description which may not otherwise qualify as prior art at the time of filing, are neither expressly or impliedly admitted as prior art against the present disclosure.

Some computer assisted surgery systems allow a human surgeon and a computerised surgical apparatus (e.g. surgical robot) to work together when performing surgery. Computer assisted surgery systems include, for example, computer-assisted medical scope or camera systems (e.g. where a computerised surgical apparatus holds and positions a medical scope (also known as a medical vision scope) such as a medical endoscope, surgical microscope or surgical exoscope while a human surgeon conducts surgery using the medical scope images), master-slave systems (comprising a master apparatus used by the surgeon to control a robotic slave apparatus) and open surgery systems in which both a surgeon and a computerised surgical apparatus autonomously perform tasks during the surgery.

A problem with such computer assisted surgery systems is there is sometimes a discrepancy between a human decision made by the human surgeon and a computer decision made by the computerised surgical apparatus. In this case, it can be difficult to know why there is a decision discrepancy and, in turn, which of the human and computer decisions is correct. There is a need for a solution to this problem.

### SUMMARY

According to the present disclosure, a computer assisted surgery system is provided that includes a computerised surgical apparatus and a control apparatus, wherein the control apparatus includes circuitry configured to: receive information indicating a first region of a surgical scene from which information is obtained by the computerised surgical apparatus to make a decision; receive information indicating a second region of the surgical scene from which information is obtained by a medical professional to make a decision; determine if there is a discrepancy between the first and second regions of the surgical scene; and if there is a discrepancy between the first and second regions of the surgical scene: perform a predetermined process based on the discrepancy.

### BRIEF DESCRIPTION OF DRAWINGS

Non-limiting embodiments and advantages of the present disclosure will be best understood by reference to the following detailed description taken in conjunction with the accompanying drawings.

FIG. 1 schematically shows a computer assisted surgery system.

FIG. 2 schematically shows a control apparatus and computerised surgical apparatus controller.

FIG. 3A schematically shows a surgical scene as viewed by a human surgeon and a surgical robot.

FIG. 3B schematically shows the surgical scene as viewed by the surgical robot after adjustment of one or more parameters.

FIG. 4 shows a surgical control method.

FIG. 5 schematically shows a first example of a computer assisted surgery system to which the present technique is applicable.

FIG. 6 schematically shows a second example of a computer assisted surgery system to which the present technique is applicable.

FIG. 7 schematically shows a third example of a computer assisted surgery system to which the present technique is applicable.

FIG. 8 schematically shows a fourth example of a computer assisted surgery system to which the present technique is applicable.

FIG. 9 schematically shows an example of an arm unit.

FIG. 10 schematically shows an example of a master console.

Like reference numerals designate identical or corresponding parts throughout the drawings.

### DESCRIPTION OF EMBODIMENTS

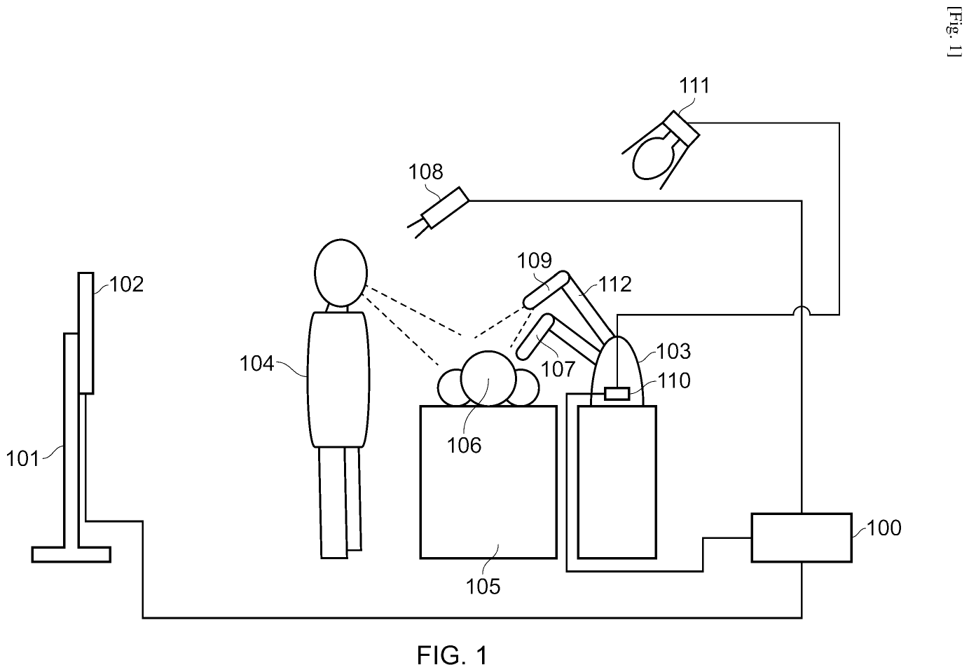

FIG. 1 shows surgery on a patient 106 using an open surgery system. The patient 106 lies on an operating table 105 and a human surgeon 104 and a computerised surgical apparatus 103 perform the surgery together.

Each of the human surgeon and computerised surgical apparatus monitor one or more parameters of the surgery, for example, patient data collected from one or more patient data collection apparatuses (e.g. electrocardiogram (ECG) data from an ECG monitor, blood pressure data from a blood pressure monitor, etc.—patient data collection apparatuses are known in the art and not shown or discussed in detail) and one or more parameters determined by analysing images of the surgery (captured by the surgeon's eyes or a camera 109 of the computerised surgical apparatus) or sounds of the surgery (captured by the surgeon's ears or a microphone (not shown) of the computerised surgical apparatus). Each of the human surgeon and computerised surgical apparatus carry out respective tasks during the surgery (e.g. some tasks are carried out exclusively by the surgeon, some tasks are carried out exclusively by the computerised surgical apparatus and some tasks are carried out by both the surgeon and computerised surgical apparatus) and make decisions about how to carry out those tasks using the monitored one or more surgical parameters.

There can sometimes be conflict in the decisions made by the surgeon and computerised surgical apparatus. For example, based on image analysis, the computerised surgical apparatus may decide an unexpected bleed has occurred in the patient and that action should be taken to stop the bleed. However, the surgeon may decide there is not an unexpected bleed and that no action needs to be taken. There is therefore a need to resolve this conflict so that the correct decision is made. The present technique helps fulfil this need by determining if there is a discrepancy in visual regions of the surgical scene paid attention to by the surgeon and computerised surgical apparatus when a decision by the computerised surgical apparatus is made and, if there is a discrepancy, controlling the computerised surgical apparatus to reassess the surgical scene. The computerised surgical apparatus makes an updated decision based on the reassessed surgical scene. This decision may be accepted or rejected by the surgeon. Information about why the computerised surgical apparatus made a particular decision may be provided to the surgeon to help them determine which decision (i.e. human decision or computer decision) is the correct one.

Although FIG. 1 shows an open surgery system, the present technique is also applicable to other computer assisted surgery systems where the computerised surgical apparatus (e.g. which holds the medical scope in a computer-assisted medical scope system or which is the slave apparatus in a master-slave system) is able to make decisions which might conflict with the surgeon's decisions. The computerised surgical apparatus is therefore a surgical apparatus comprising a computer which is able to make a decision about the surgery using one or more monitored parameters of the surgery. As a non-limiting example, the computerised surgical apparatus 103 of FIG. 1 is a surgical robot capable of making decisions and undertaking autonomous actions based on images captured by the camera 109.

The robot 103 comprises a controller 110 and one or more surgical tools 107 (e.g. movable scalpel, clamp or robotic hand). The controller 110 is connected to the camera 109 for capturing images of the surgery, to a movable camera arm 112 for adjusting the position of the camera 109 and to adjustable surgical lighting 111 which illuminates the surgical scene and has one or more adjustable lighting parameters such as brightness and colour. For example, the adjustable surgical lighting comprises a plurality of light emitting diodes (LEDs, not shown) of different respective colours. The brightness of each LED is individually adjustable (by suitable control circuitry (not shown) of the adjustable surgical lighting) to allow adjustment of the overall colour and brightness of light output by the LEDs. The controller 110 is also connected to a control apparatus 100. The control apparatus 100 is connected to another camera 108 for capturing images of the surgeon's eyes for use in gaze tracking and to an electronic display 102 (e.g. liquid crystal display) held on a stand 102 so the electronic display 102 is viewable by the surgeon 104 during the surgery. The control apparatus 100 compares the visual regions of the surgical scene paid attention to by the surgeon 104 and robot 103 to help resolve conflicting surgeon and computer decisions according to the present technique.

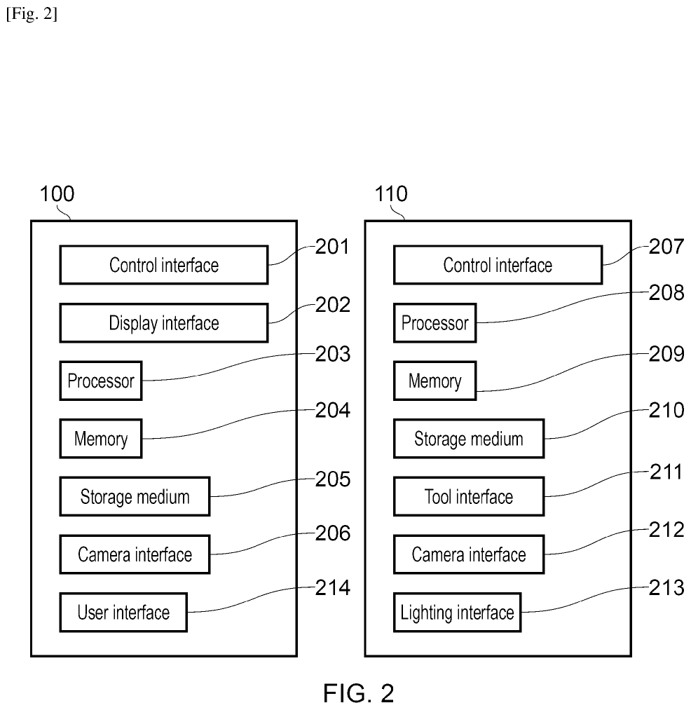

FIG. 2 shows some components of the control apparatus 100 and controller 110.

The control apparatus 100 comprises a control interface 201 for sending electronic information to and/or receiving electronic information from the controller 110, a display interface 202 for sending electronic information representing information to be displayed to the electronic display 102, a processor 203 for processing electronic instructions, a memory 204 for storing the electronic instructions to be processed and input and output data associated with the electronic instructions, a storage medium 205 (e.g. a hard disk drive, solid state drive or the like) for long term storage of electronic information, a camera interface 206 for receiving electronic information representing images of the surgeon's eyes captured by the camera 108 and a user interface 214 (e.g. comprising a touch screen, physical buttons, a voice control system or the like). Each of the control interface 201, display interface 202, processor 203, memory 204, storage medium 205, camera interface 206 and user interface 214 are implemented using appropriate circuitry, for example. The processor 203 controls the operation of each of the control interface 201, display interface 202, memory 204, storage medium 205, camera interface 206 and user interface 214.

The controller 110 comprises a control interface 207 for sending electronic information to and/or receiving electronic information from the control apparatus 100, a processor 208 for processing electronic instructions, a memory 209 for storing the electronic instructions to be processed and input and output data associated with the electronic instructions, a storage medium 210 (e.g. a hard disk drive, solid state drive or the like) for long term storage of electronic information, a tool interface 211 for sending electronic information to and/or receiving electronic information from the one or more surgical tools 107 of the robot 103 to control the one or more surgical tools, a camera interface 212 for receiving electronic information representing images of the surgical scene captured by the camera 109 and to send electronic information to and/or receive electronic information from the camera 109 and movable camera arm 112 to control operation of the camera 109 and movement of the movable camera arm 112 and a lighting interface 213 to send electronic information to and/or receive electronic information from the adjustable surgical lighting 111 to adjust the one or more surgical lighting parameters. Each of the control interface 107, processor 208, memory 209, storage medium 210, tool interface 211, camera interface 212 and lighting interface 213 are implemented using appropriate circuitry, for example. The processor 208 controls the operation of each of the control interface 107, memory 209, storage medium 210, tool interface 211, camera interface 212 and lighting interface 213. The controller 110 controls the robot 103 to make decisions and undertake autonomous surgical actions (e.g. using the one or more surgical tools 107) according to the capabilities of the robot 103.

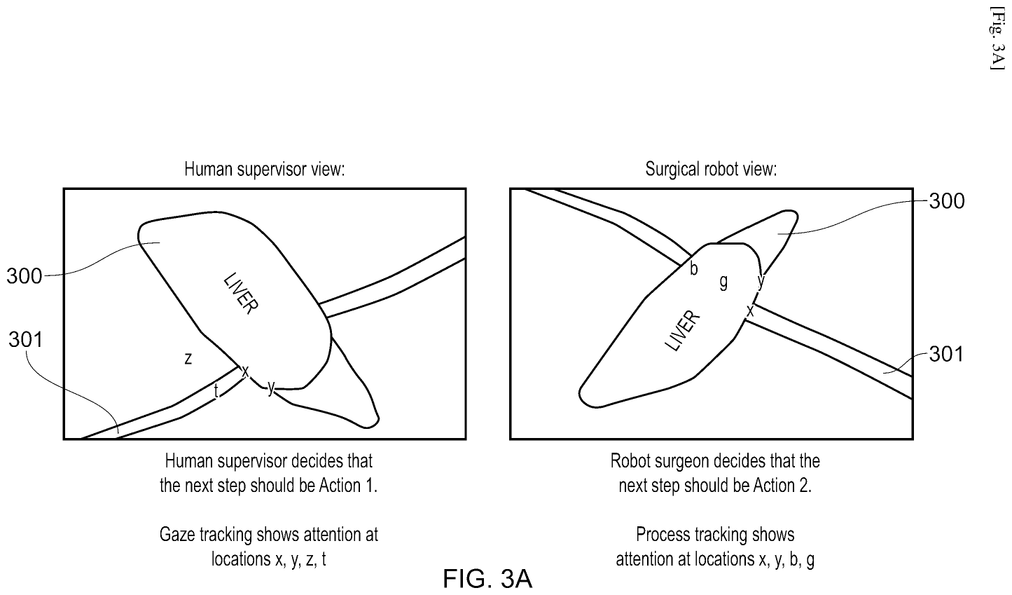

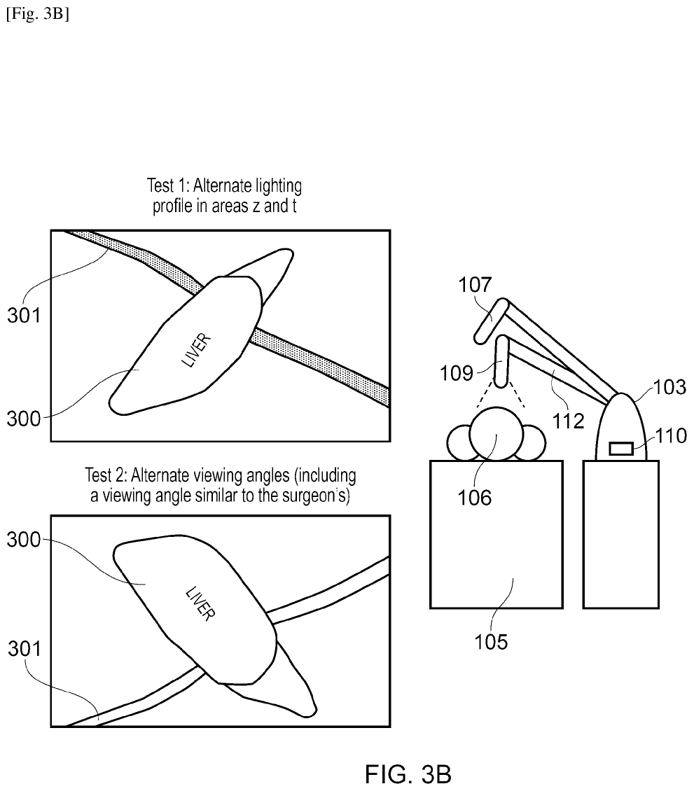

FIG. 3A shows an example situation in which the surgeon 104 and robot 103 make a conflicting decision. Both the surgeon (with their eyes) and the robot 103 (with camera 109) are viewing the surgical scene comprising the patient's liver 300 and a blood vessel 301. The surgeon and robot are viewing the surgical scene from different angles. The control apparatus 100 monitors the regions of the scene the surgeon and the robot are paying attention to (i.e. the regions of the scene used by the surgeon and robot to obtain information (in particular, visual information) about the scene to make decisions). When the robot makes a decision, the control apparatus receives information indicating the decision. In response to this, the regions of the scene paid attention to by the surgeon and robot for a predetermined time period up to when the robot decision was made are compared.

The regions the surgeon is paying attention to are determined by tracking the surgeon's gaze. Any suitable known gaze tracking technique may be used. In this example, the surgeon's gaze is tracked by tracking the location of the surgeon's eyes in the images captured by the camera 108. Prior to surgery, a calibration procedure is undertaken in which the surgeon is instructed to look at each of a plurality of regions of the scene. When the surgeon is looking at each region, an image of the surgeon's eyes is captured and the position of the surgeon's eyes in the image is detected (e.g. using any suitable known object detection technique). This allows a mapping between the position of the surgeon's eyes in images captured by the camera 108 and the region of the scene the surgeon is looking at to be determined. Information indicative of this mapping is stored in the storage medium 205. During surgery, this information is then used by the control apparatus 100 to determine the region of the scene being looked at by the surgeon (i.e. the surgeon's gaze location) at any given time. In FIG. 3A, the control apparatus 100 determines the surgeon's gaze was primarily in regions x, y, z and t of the scene (e.g. based on the amount of time the surgeon's gaze rests on these regions relative to other regions of the scene) during the predetermined time period up to when the robot decision was made. These are determined to be the regions the surgeon was paying attention to.

The regions the robot is paying attention to are determined by analysing the regions of images captured by the camera 109 which are most likely to influence a decision made by the robot using those images. There are various known techniques for doing this. An example is NPL1 which allows an artificial intelligence (AI) attention heat map to be generated which indicates regions of a captured image which most influence the output of an image classification convolutional neural network (CNN). Information indicative of a mapping between regions of images captured by the camera 109 and regions of the scene is stored in the storage medium 205. During surgery, this information is then used by the control apparatus 100 to determine the region of the scene being paid attention to by the robot when a decision is made (each decision made by the robot being based on a classification of an image captured by the camera 109, for example). The control apparatus 100 determines the regions b, g, y and x as the regions the robot is paying attention to (e.g. based on the amount of time the robot pays attention to these regions relative to other regions of the scene) during the predetermined time period up to when the robot decision was made.

The control apparatus 100 therefore recognises a discrepancy between the regions x, y, z and t paid attention to by the surgeon 104 and the regions x, y, b and g paid attention to by the robot 103. Comparison of the regions is possible by ensuring the regions of the scene mapped to respective eye positions of the surgeon in images captured by the camera 108 are the same as the regions of the scene mapped to respective regions of images captured by the camera 109 and used by the robot for classification and decision making. The discrepancy in this case indicates the computer decision made by the robot might be different to a human decision made by the surgeon.

In an embodiment, a discrepancy occurs when a region of the scene paid attention to by the surgeon only is spatially separated from a region of the scene paid attention to by the robot only by more than a predetermined threshold. The threshold may be chosen in advance based on an acceptable discrepancy for the surgery concerned. A larger threshold allows greater deviation in the regions paid attention to by the robot and surgeon before a discrepancy is registered (this is appropriate when completing surgery quickly without unnecessary interruption is most important, for example). A smaller threshold allows less deviation in the regions paid attention to by the robot and surgeon before a discrepancy is registered (this is appropriate for intricate surgery which must be completed correctly even if it takes a long time, for example).

In this case, the computer decision is different to the human decision. The surgeon has decided the next step of the surgery should be Action 1 (e.g. proceed to the next stage of the predefined surgical procedure). However, the robot has decided the next step of the surgery should be Action 2 (e.g. take action to alleviate a detected bleed of the blood vessel 301). The discrepancy could be the result of (1) an error made by the robot which is paying attention to regions x, y, b and g (which include mainly the liver 300 but not the blood vessel 301) while the surgeon is paying attention to regions x, y, z and t (with region t including the blood vessel 301). Alternatively, the discrepancy could be the result of (2) an error made by the surgeon (who, although paying attention to region t including the blood vessel may not have noticed the bleed for reasons of human error).

The control apparatus 100 makes it possible to determine which of (1) and (2) is more likely by controlling the robot 103 to pay attention to the regions z and t previously paid attention to by the surgeon 104 but not the robot and to make an updated computer decision based on these regions. The robot is controlled to pay attention to the previously overlooked regions z and t by adjusting one or more suitable parameters used in the image processing by the robot. For example, if the robot uses an image classification CNN to classify images captured by the camera 109 and make decisions based on those classifications, the weightings of the CNN may be adjusted to increase the influence of regions z and t in the classification process.

In order to improve the accuracy of the updated decision, the control apparatus 100 may control the robot 103 to adjust one or more further parameters used by the robot when the initial decision was made. For example, the robot is controlled to adjust one or more lighting parameters of the adjustable surgical lighting 111 and/or to adjust the position of the camera 109 using the movable camera support arm 112. This helps reduce the effect of any visual conditions of the surgical scene as viewed through the camera 109 which may have hindered the robot paying attention to and/or making a decision based on the regions z and t when the initial decision was made.

In an example, object recognition in images captured by the camera 109 in the regions z and t may be used to determine suitable values of the one or more lighting parameters. For example, since the region t includes the blood vessel, suitable values of the one or more lighting parameters may be chosen which help visually distinguish objects with the colour of the blood vessel 301 from objects with other colours in the captured images. This is achieved by changing the light to a colour which more accurately matches that of the blood vessel 301 (e.g. a shade of red which more accurately matches the shade of red of the blood vessel 301) and/or increasing the brightness of the light. This is shown in FIG. 3B. The more highly distinguished blood vessel allows the robot to more accurately determine if there has indeed been a bleed of the blood vessel 301.

In an example, the camera is positioned 109 to have a viewing angle similar to that of the surgeon when the initial decision was made. This is also shown in FIG. 3B, which shows both the repositioned camera and the image of the scene captured by the repositioned camera (which is similar in perspective to the surgeon's view of the scene of FIG. 3A). This allows the robot to see the surgical scene from a similar perspective to the surgeon, who may have a better view of the part of the scene relevant to the decision (e.g. the part of the blood vessel 301 the robot determined was bleeding). Again, this allows the robot to more accurately determine if there has indeed been a bleed of the blood vessel 301. In an example, the control apparatus 100 monitors the position of the surgeon 104 in the operating theatre and controls the camera 109 to be repositioned close to (e.g. within a predetermined distance of) the monitored position of the surgeon when the initial decision was made. The position of the surgeon in the operating theatre is known, for example, by recognising the surgeon in images captured by a further camera (not shown) which captures images of the entire operating theatre (e.g. using any suitable known object detection technique). Positions in the captured images are mapped to positions in the operating theatre (information indicative of this mapping is stored in the storage medium 205, for example), thereby allowing the position of the surgeon in the operating theatre to be determined based on their detected position in the captured images. The camera 109 is then moved to a position within a predetermined distance of the detected position of the surgeon. This is possible, for example, by the storage medium 205 storing information indicative of a mapping between positions in the operating theatre and one or more parameters of the movable camera arm 112 which determine the position of the camera 109. In embodiment, the camera 109 is moved to within a predetermined distance range from the surgeon, with a minimum of the distance range being set to avoid collision of the surgeon with the camera 109 and a maximum of the distance range being set so that the perspective of the camera 109 sufficiently approximates that of the surgeon when the initial decision was made by the robot.

Once the robot 103 has made the updated decision and provided it to the control apparatus 100, the control apparatus outputs visual information indicating the updated decision to the electronic display 102 to be viewed by the surgeon. If the updated decision is the same as the initial decision (i.e. Action 2) even after looking at the previously overlooked regions z and t with, optionally, different light and/or a different camera angle, scenario (2) (i.e. surgeon error) is more likely. Alternatively, if the updated decision is different to the initial decision (e.g. Action 1, as initially determined by the surgeon), scenario (1) (i.e. robot error) is more likely. The control apparatus 100 controls action to be taken by the robot in response to the updated decision (e.g. perform actions associated with proceeding to the next stage of the predefined surgical procedure if the updated decision is Action 1 or perform actions associated with alleviating a detected bleed of the blood vessel 301 if the updated decision is Action 2) upon receiving confirmation from the surgeon. The confirmation is received from the surgeon via the user interface 214. The present technique thus allows improved validation of computer decisions made by surgical robot 103. This is because, if the regions of the surgical scene being paid attention to by the surgeon differ from those of the robot when a computer decision is made by the robot, additional information is sought by the robot and, if necessary, the computer decision is updated before notifying the surgeon of that decision. The computer decision is therefore more likely to be accurate.

In an embodiment, information indicating a computer decision made by the robot 103 is displayed as a message on the electronic display 102 together with options for the surgeon to either (a) accept the decision (in which case the robot carries out any actions associated with the computer decision, e.g. actions to stop a bleed of blood vessel 301) or (b) reject the decision (in which case the robot continues to carry out any actions they would have done if the computer decision had not been made, e.g. actions associated with the next stage of the predefined surgical procedure). The surgeon selects either (a) or (b) via the user interface 214. In an example, the user interface 214 comprises a microphone and voice recognition software which accepts the computer decision when it detects the surgeon say “accept” or rejects the computer decision when it detects the surgeon say “reject”. When the displayed computer decision is an updated computer decision, the control apparatus 100 may control the electronic display 102 to display information indicating that the decision is an updated decision together with information indicating the initial decision and information about how the updated decision was made. The information indicating how the updated decision was made includes, for example, the different regions of the surgical scene paid attention to by the surgeon and robot when the initial decision was made (e.g. by displaying the images of the scene of FIG. 3A (as captured by camera 109) overlaid with information indicating the regions paid attention to by each of the surgeon and robot when the initial decision was made, optionally with the regions paid attention to by the robot but not the surgeon indicated as such (via suitable highlighting or the like) so the surgeon is able to quickly ascertain the regions in which they may have missed something) and information indicating the one or more adjusted parameters (e.g. lighting colour and/or brightness and/or the angle of camera 109, optionally including the alternative lighting and/or viewing angle images captured by the camera 109 as shown in FIG. 3B) used to make the updated decision.

This allows the surgeon to obtain a better understanding of the robot's decision making process which, in turn, allows them to make a more informed decision about whether to accept the updated decision provided by the robot. For example, in FIG. 3A, if the updated decision of the robot is that a bleed of blood vessel 301 has occurred and should therefore be quickly alleviated, the surgeon will be able to see from the information displayed by the electronic display 102 that the robot was initially not paying attention to region t (including the blood vessel 301) paid attention to by the surgeon but that, in coming to the updated decision, the robot has paid attention to this region and has furthermore adjusted the lighting and position of camera 109 to obtain as much information about this region as possible. The surgeon can therefore look again at region t carefully and accept or reject the updated decision from the robot. The decision making process of the robot is thus more transparent to the surgeon which, ultimately, allows the surgeon to make a more informed decision when accepting or rejecting a decision made by the robot.

Any other parameter may be adjusted (instead of or in addition to lighting and/or viewing angle parameters) when determining the updated computer decision from the robot 103. For example, an adversarial pixel change (or another change designed to affect an artificial intelligence-based image classification process by changing the value of one or more pixels) in a region of an image captured by the camera 109 and used by the robot to make the initial decision (e.g. the image regions corresponding to the regions z and t in the scene not initially paid attention to by the robot) may be made prior to the image classification for determining the updated decision. Multiple such changes could be made and the resulting most common classification (and corresponding most common updated decision) provided as the updated decision. This helps to alleviate the effects of noise in the updated decision. Another example of an adjustable parameter is the type of image taken (e.g. the camera 109 may comprise multiple image sensors capable of capturing images using different types of electromagnetic radiation (e.g. visible light, ultraviolet and infrared) and, upon determining a discrepancy in the attention regions of the surgeon 104 and robot 103, the control apparatus 100 controls the camera 109 to capture an image using a different sensor to that previously used and to perform image classification on the newly captured image). In the case of a discrepancy in the surgeon and robot attention regions, the control apparatus 100 may also control the robot to recalibrate the image sensor(s) used by the camera 109 to capture images of the surgical scene.

More generally, multiple candidate updated decisions may be made with different respective sets of adjusted parameters. The most common candidate updated decision is then output as the updated decision to be accepted or rejected by the surgeon 104. The extent to which parameters are adjusted (e.g. how many parameters are adjusted and how many adjustments are made to each parameter) and therefore how many candidate updated decisions there are may depend on one or more factors, such as the extent to which the attention of the surgeon 104 and robot 103 differs, the risk of the wrong decision being made, the respective viewing angles of the surgeon and robot and/or the type of imaging used by the robot in making the decision. For example, fewer parameters may be adjusted and/or fewer adjustments per parameter may be made to produce a lower number of candidate updated decisions when there is greater overlap in the attention regions of the robot and surgeon (e.g. when the percentage of regions paid attention to by the surgeon which are also paid attention to by the robot exceeds a predetermined amount) and/or when the risk of the wrong decision being made is lower (e.g. below a predetermined threshold). Conversely, more parameters may be adjusted and/or more adjustments per parameter may be made to produce a higher number of candidate updated decisions when there is lesser overlap in the attention regions of the robot and surgeon (e.g. when the percentage of regions paid attention to by the surgeon which are also paid attention to by the robot is less than a predetermined amount) and/or when the risk of the wrong decision being made is higher (e.g. above a predetermined threshold). This risk associated with making the wrong decision for each computer decision which may be made by the robot is stored (e.g. as a numerical value which is higher the higher the risk) in the storage medium 205, for example. The extent to which parameters were adjusted to determine the candidate updated decisions and, ultimately, the updated decision may be displayed to the surgeon on electronic display 102 along with the updated decision and options to accept or reject the updated decision.

Although the above description considers a surgeon, the present technique is applicable to any human supervisor in the operating theatre (e.g. another medical professional such as an anaesthetist, nurse, etc.) whose attention on regions of the surgical scene may be monitored (e.g. through gaze tracking) and whose decisions may conflict with those made by a computerised surgical apparatus.

The image classification used by the robot to make a decision uses any suitable feature(s) of the image to make the classification. For example, the image classification may recognise predefined objects in the image (e.g. particular organs or surgical tools) or may make use of image colouration, topography or texture. Any suitable known technique may be used in the image classification process.

Although the computer decision in the above example relates to whether or not an action should take place (e.g. stop bleed or continue with the next predefined step of the surgery, make an incision or not make an incision, etc.), the computer decision may be another type of decision such as deciding that a particular object is a specific organ or deciding whether a particular tissue region is normal or abnormal. It will be appreciated that the present technique could be applied to any type of decision.

Although in the above example the gaze of the surgeon is tracked using images captured by the camera 108, any other suitable gaze tracking technology (e.g. a head mountable device worn by the surgeon which directly tracks eye movement using electrooculography (EOG)) may be used. Similarly, the electronic display 102 may be part of a head mountable display (HMD) or the like worn by the surgeon rather than a separate device (e.g. monitor or television) viewable by the surgeon (as shown in FIG. 1).

In an embodiment, the control apparatus 100 determines a confidence rating of the updated decision indicating a likelihood the updated decision is the correct decision and causes the electronic display 102 to display the confidence rating with the updated decision. The confidence rating may be determined using any suitable method and may be calculated by the processor 208 of the robot controller 110 as part of the image classification process (in this case, the confidence value is provided to the control apparatus 100 for subsequent display on the electronic display 102). In the case the initial decision is also associated with a confidence value, the control apparatus 100 may indicate the confidence values or both the initial and updated decisions or, at least, may indicate whether the confidence value of the updated decision is higher or lower than that of the initial decision. This provides the surgeon with further information to help them decide whether to accept or reject the updated decision.

In some embodiments (e.g. computer-assisted medical scope systems or master-slave systems), the robot still makes a decision for a certain stage or activity during the surgery, but this decision is used to inform the surgeon's actions rather than for the robot to directly carry out an action independently. When using a computer assisted surgery system, the surgeon may view the surgical scene by looking at a captured image of the surgical scene (e.g. captured by a medical scope or by a camera of a surgical robot) rather than the surgical scene itself. In this case, the surgeon's gaze over the captured image rather than the surgical scene itself is used to determine the attention regions of the surgeon (which are then compared directly with the attention regions of the robot on the same image).

In one example, additional imaging modalities are available to the robot to help make a computer decision. For example, a medical scope image viewed by the surgeon may typically be an RGB image that is easy to interpret while other modalities (e.g. using different wavelengths to provide hyperspectral imaging) obtained from other imaging equipment connected to the robot (not shown) may be available but less intuitive to the surgeon.

Comparison of the human and robot attention regions allows the combination of more intuitive images (e.g. RGB images) and less intuitive images (e.g. hyperspectral images) of the scene to be used more effectively. The example surgical sequence below (which is an embodiment) illustrates this:

1. The surgeon is carrying out surgery with assistance from a robot (e.g. a robot in a computer-assisted medical scope system or a master-slave surgical robot).

2. The robot assesses the surgical scene using an RGB image as previously described, but also incorporates one or more other imaging modalities to assist in making a decision.

3. Simultaneously, the surgeon assesses the surgical scene to make the human decision but uses only an RGB viewing mode to do so.

4. A discrepancy in the human and robot attention regions of the RGB image is detected.

There therefore may be a discrepancy between the human and robot decisions.

5. In response to the discrepancy, one or more parameters used by the robot to make the decision are adjusted for each of the RGB image and the one or more other imaging modalities.

6. The robot makes an updated decision based on the adjusted parameters. The control apparatus 100 outputs information indicating the updated decision for display on the electronic display 102, together with information (e.g. one or more images) determined from the alternate viewing modalities used by the robot to make the updated decision.

Thus, the surgeon is provided with the more intuitive images (e.g. RGB images) all the time and with the less intuitive images (e.g. hyperspectral images) only when an updated decision is made by the robot. This provides an improved balance between presenting less, intuitive information and more, less intuitive information.

In this example, the region(s) of the scene the robot is paying attention to (e.g. as determined in step 4) may also be assessed using images of the scene captured by the robot using another modality (e.g. hyperspectral images). In this case, the regions of the scene to which the robot and surgeon may pay attention are defined in the same way. However, the surgeon is viewing the scene via an RGB image and the robot is viewing the scene via a different image modality.

A discrepancy in attention region may therefore occur because an event in the scene is more readily detectable by the robot using the different image modality than the surgeon using the RGB image.

In one example, the field of view shown to the surgeon during medical scope or master-slave operated surgery is narrower than that visible to the robot due to limitations imposed by monitor(s) displaying the medical scope images. For example, the edges of the medical scope image may be cropped to facilitate the surgeon's view of the surgical scene. In this case, additional information may be available to the robot that is not available to the surgeon. A problem is therefore when to change the camera view to show the surgeon the otherwise cropped region of the image. The present technique alleviates this problem by indicating the camera view should be changed when one or more of the robot attention regions are in the cropped region of the image. The example surgical sequence below (which is an embodiment) illustrates this:

1. During surgery, the robot makes a decision using information in a captured image of the surgical scene. The image includes a cropped region not visible to the surgeon.

2. When one or more of the robot attention regions is in the cropped region, the control apparatus 100 causes the display apparatus 102 to display information indicating this to the surgeon. The camera view may then be altered (e.g. manually by the surgeon or scopist or automatically by the robot 103 under control of the control apparatus 100) to show the surgeon the cropped region of the image paid attention to by the robot. An appropriate part of the cropped region (in which an event may have occurred but which they surgeon would not have otherwise seen) is therefore shown to the surgeon, thereby providing them with more information with which to make a decision.

In one example, during surgery using an open or master-slave surgery system, the surgeon may make decisions using only captured RGB images. Without other information (e.g. haptic feedback or the like), no indication of force or texture is provided to the surgeon. On the other hand, the robot may be able to take into account such information (e.g. based on information output by one or more accelerometers (not shown), force sensors (e.g. piezoelectric devices) or the like comprised by one or more tools of the robot). The force information is not available to the surgeon. For example, the robot may be able to determine when a tool has become stuck on tissue within the body cavity using the force information (e.g. if the output of a force sensor of the tool exceeds a predetermined threshold or, more generally, is outside a predetermined range, or if the output of an accelerometer of the tool indicates a deceleration of the tool with a magnitude exceeding a predetermined threshold, or more generally, outside a predetermined range). The present technique allows the surgeon to be made aware of this information, if appropriate. The example surgical sequence below (which is an embodiment) illustrates this:

1. The robot makes a decision using an RGB image as previously described, but also assesses information indicative of force and/or motion data of a tool of the robot. This information is not available to the surgeon. This information is extracted by, for example:

a. Using visual assessment of the interaction between the robot tool and the tissue (e.g. using a suitable known object detection method, image classification method or the like on the RGB image when the RGB image includes robot tool); and/or

b. Using one or more additional sensors comprised by the robot tool (e.g. accelerometers, force sensors or the like).

Using this information, the robot is able to determine unusual force and/or motion data (e.g. if the force exceeds a predetermined threshold or the speed of the robot tool falls below a predetermined threshold) and make a decision using this data (e.g. to pause the surgical procedure and alert the surgeon to investigate the tool, which may have become stuck).

2. When a discrepancy between the human and robot attention regions is detected and relates to the unusual force and/or motion data (e.g. if the human attention region(s) indicate the human is unlikely to be paying attention to a region of the RGB image including the robot tool for which unusual force and/or motion data has been recorded), the control apparatus 100 controls the electronic display 102 to output information indicating the region in which unusual force and/or motion was detected (e.g. as information overlaid on the RGB image). An indication of the unusual force and/or motion may be displayed (this information being received by the controller 110 of the robot 103 via the tool interface 211).

The present technique therefore enables improved human-robot collaboration in a surgical setting by enabling intuitive supervision of the robot system and improved interpretability of the robot's decisions. In particular, intuitive human supervision of the robot system is enabled through the comparison of attention regions of the human and robot which may be determined as required to validate a robot decision. Increased interpretability of robot decisions is enabled through the output of visual information (e.g. images of the surgical scene overlaid with information indicating the attention regions of the human and robot, adjusted parameters used to generated an updated robot decision, etc.) which a human supervisor easily understands. This provides information to the human about how the robot has made a certain decision. In addition, increased opportunities for ad-hoc training and calibration of a computer assisted surgery is enabled. For example, differences in the attention regions of the human and robot may be used to identify deficiencies in the decision-making protocols or imaging processes of the robot.

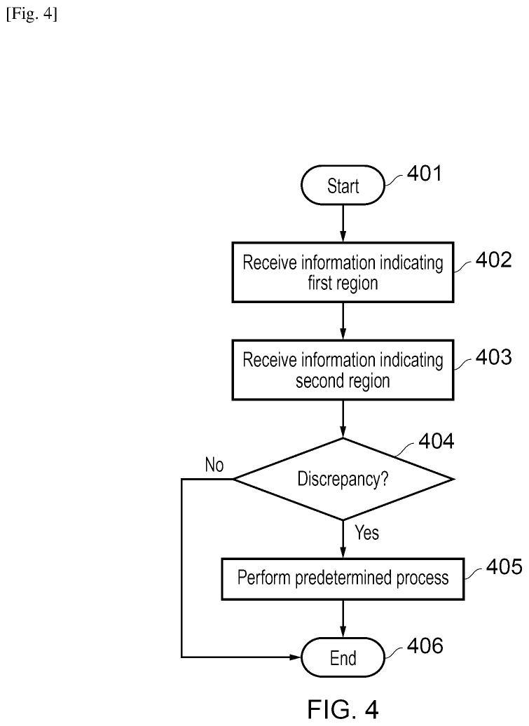

FIG. 4 shows a flow chart showing a method carried out by the control apparatus 100 according to an embodiment.

The method starts at step 401.

At step 402, the control interface 201 receives information indicating a first region (e.g. regions x, y, b and/or g) of the surgical scene to which a computerised surgical apparatus (e.g. robot 103) is paying attention. The received information is information indicating an AI attention map, for example.

At step 403, the camera interface 206 receives information indicating a second region (e.g. regions x, y, z and/or t) of the surgical scene to which a human (e.g. surgeon 104) is paying attention. The received information is information indicating the position of the humans eyes for use in gaze tracking, for example.

At step 404, the processor 203 determines if there is a discrepancy in the first and second regions.

If there is no discrepancy, the method ends at step 406.

If there is a discrepancy, the method proceeds to step 405 in which a predetermined process is performed based on the discrepancy. The predetermined process comprises, for example, controlling the computerised surgical apparatus to make an updated decision based on the discrepancy (e.g. by paying attention to the second region and/or adjusting one or more parameters used by the computerised surgical apparatus to determine the first region), to change the camera view (or indicate the camera view should be changed) to allow the human to see a previously cropped region of an image of the surgical scene or to indicate a value of an operating parameter of a surgical tool which falls outside a predetermined range, as detailed above. The method then ends at step 406.

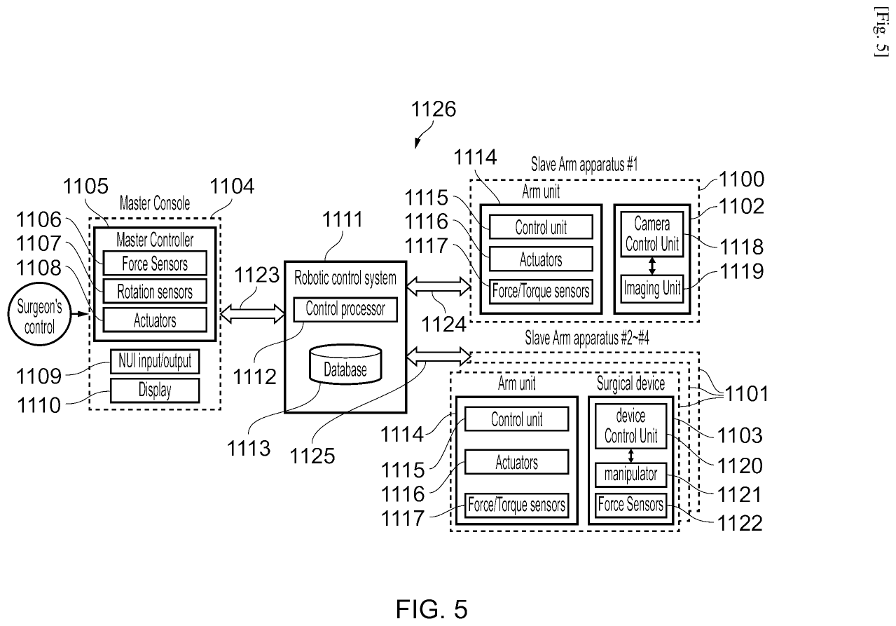

FIG. 5 schematically shows an example of a computer assisted surgery system 1126 to which the present technique is applicable. The computer assisted surgery system is a master-slave system incorporating an autonomous arm 1100 and one or more surgeon-controlled arms 1101. The autonomous arm holds an imaging device 1102 (e.g. a surgical camera or medical vision scope such as a medical endoscope, surgical microscope or surgical exoscope). The one or more surgeon-controlled arms 1101 each hold a surgical device 1103 (e.g. a cutting tool or the like). The imaging device of the autonomous arm outputs an image of the surgical scene to an electronic display 1110 viewable by the surgeon. The autonomous arm autonomously adjusts the view of the imaging device whilst the surgeon performs the surgery using the one or more surgeon-controlled arms to provide the surgeon with an appropriate view of the surgical scene in real time.

The surgeon controls the one or more surgeon-controlled arms 1101 using a master console 1104. The master console includes a master controller 1105. The master controller 1105 includes one or more force sensors 1106 (e.g. torque sensors), one or more rotation sensors 1107 (e.g. encoders) and one or more actuators 1108. The master console includes an arm (not shown) including one or more joints and an operation portion. The operation portion can be grasped by the surgeon and moved to cause movement of the arm about the one or more joints. The one or more force sensors 1106 detect a force provided by the surgeon on the operation portion of the arm about the one or more joints. The one or more rotation sensors detect a rotation angle of the one or more joints of the arm. The actuator 1108 drives the arm about the one or more joints to allow the arm to provide haptic feedback to the surgeon. The master console includes a natural user interface (NUI) input/output for receiving input information from and providing output information to the surgeon. The NUI input/output includes the arm (which the surgeon moves to provide input information and which provides haptic feedback to the surgeon as output information). The NUI input/output may also include voice input, line of sight input and/or gesture input, for example. The master console includes the electronic display 1110 for outputting images captured by the imaging device 1102.

The master console 1104 communicates with each of the autonomous arm 1100 and one or more surgeon-controlled arms 1101 via a robotic control system 1111. The robotic control system is connected to the master console 1104, autonomous arm 1100 and one or more surgeon-controlled arms 1101 by wired or wireless connections 1123, 1124 and 1125. The connections 1123, 1124 and 1125 allow the exchange of wired or wireless signals between the master console, autonomous arm and one or more surgeon-controlled arms.

The robotic control system includes a control processor 1112 and a database 1113. The control processor 1112 processes signals received from the one or more force sensors 1106 and one or more rotation sensors 1107 and outputs control signals in response to which one or more actuators 1116 drive the one or more surgeon controlled arms 1101. In this way, movement of the operation portion of the master console 1104 causes corresponding movement of the one or more surgeon controlled arms.

The control processor 1112 also outputs control signals in response to which one or more actuators 1116 drive the autonomous arm 1100. The control signals output to the autonomous arm are determined by the control processor 1112 in response to signals received from one or more of the master console 1104, one or more surgeon-controlled arms 1101, autonomous arm 1100 and any other signal sources (not shown). The received signals are signals which indicate an appropriate position of the autonomous arm for images with an appropriate view to be captured by the imaging device 1102. The database 1113 stores values of the received signals and corresponding positions of the autonomous arm.

For example, for a given combination of values of signals received from the one or more force sensors 1106 and rotation sensors 1107 of the master controller (which, in turn, indicate the corresponding movement of the one or more surgeon-controlled arms 1101), a corresponding position of the autonomous arm 1100 is set so that images captured by the imaging device 1102 are not occluded by the one or more surgeon-controlled arms 1101.

As another example, if signals output by one or more force sensors 1117 (e.g. torque sensors) of the autonomous arm indicate the autonomous arm is experiencing resistance (e.g. due to an obstacle in the autonomous arm's path), a corresponding position of the autonomous arm is set so that images are captured by the imaging device 1102 from an alternative view (e.g. one which allows the autonomous arm to move along an alternative path not involving the obstacle).

It will be appreciated there may be other types of received signals which indicate an appropriate position of the autonomous arm.

The control processor 1112 looks up the values of the received signals in the database 1112 and retrieves information indicating the corresponding position of the autonomous arm 1100. This information is then processed to generate further signals in response to which the actuators 1116 of the autonomous arm cause the autonomous arm to move to the indicated position.

Each of the autonomous arm 1100 and one or more surgeon-controlled arms 1101 includes an arm unit 1114. The arm unit includes an arm (not shown), a control unit 1115, one or more actuators 1116 and one or more force sensors 1117 (e.g. torque sensors). The arm includes one or more links and joints to allow movement of the arm. The control unit 1115 sends signals to and receives signals from the robotic control system 1111.

In response to signals received from the robotic control system, the control unit 1115 controls the one or more actuators 1116 to drive the arm about the one or more joints to move it to an appropriate position. For the one or more surgeon-controlled arms 1101, the received signals are generated by the robotic control system based on signals received from the master console 1104 (e.g. by the surgeon controlling the arm of the master console). For the autonomous arm 1100, the received signals are generated by the robotic control system looking up suitable autonomous arm position information in the database 1113.

In response to signals output by the one or more force sensors 1117 about the one or more joints, the control unit 1115 outputs signals to the robotic control system. For example, this allows the robotic control system to send signals indicative of resistance experienced by the one or more surgeon-controlled arms 1101 to the master console 1104 to provide corresponding haptic feedback to the surgeon (e.g. so that a resistance experienced by the one or more surgeon-controlled arms results in the actuators 1108 of the master console causing a corresponding resistance in the arm of the master console). As another example, this allows the robotic control system to look up suitable autonomous arm position information in the database 1113 (e.g. to find an alternative position of the autonomous arm if the one or more force sensors 1117 indicate an obstacle is in the path of the autonomous arm).

The imaging device 1102 of the autonomous arm 1100 includes a camera control unit 1118 and an imaging unit 1119. The camera control unit controls the imaging unit to capture images and controls various parameters of the captured image such as zoom level, exposure value, white balance and the like. The imaging unit captures images of the surgical scene. The imaging unit includes all components necessary for capturing images including one or more lenses and an image sensor (not shown). The view of the surgical scene from which images are captured depends on the position of the autonomous arm.

The surgical device 1103 of the one or more surgeon-controlled arms includes a device control unit 1120, manipulator 1121 (e.g. including one or more motors and/or actuators) and one or more force sensors 1122 (e.g. torque sensors).

The device control unit 1120 controls the manipulator to perform a physical action (e.g. a cutting action when the surgical device 1103 is a cutting tool) in response to signals received from the robotic control system 1111. The signals are generated by the robotic control system in response to signals received from the master console 1104 which are generated by the surgeon inputting information to the NUI input/output 1109 to control the surgical device. For example, the NUI input/output includes one or more buttons or levers comprised as part of the operation portion of the arm of the master console which are operable by the surgeon to cause the surgical device to perform a predetermined action (e.g. turning an electric blade on or off when the surgical device is a cutting tool).

The device control unit 1120 also receives signals from the one or more force sensors 1122. In response to the received signals, the device control unit provides corresponding signals to the robotic control system 1111 which, in turn, provides corresponding signals to the master console 1104. The master console provides haptic feedback to the surgeon via the NUI input/output 1109. The surgeon therefore receives haptic feedback from the surgical device 1103 as well as from the one or more surgeon-controlled arms 1101. For example, when the surgical device is a cutting tool, the haptic feedback involves the button or lever which operates the cutting tool to give greater resistance to operation when the signals from the one or more force sensors 1122 indicate a greater force on the cutting tool (as occurs when cutting through a harder material, e.g. bone) and to give lesser resistance to operation when the signals from the one or more force sensors 1122 indicate a lesser force on the cutting tool (as occurs when cutting through a softer material, e.g. muscle). The NUI input/output 1109 includes one or more suitable motors, actuators or the like to provide the haptic feedback in response to signals received from the robot control system 1111.

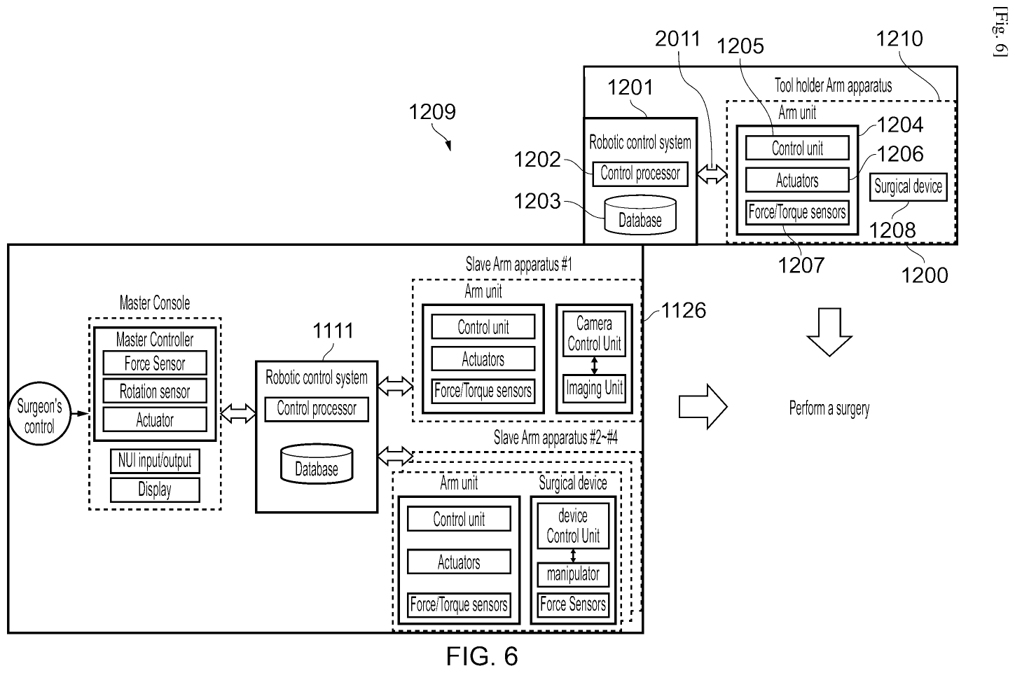

FIG. 6 schematically shows another example of a computer assisted surgery system 1209 to which the present technique is applicable. The computer assisted surgery system 1209 is a surgery system in which the surgeon performs tasks via the master-slave system 1126 and a computerised surgical apparatus 1200 performs tasks autonomously.

The master-slave system 1126 is the same as FIG. 5 and is therefore not described. The master-slave system may, however, be a different system to that of FIG. 5 in alternative embodiments or may be omitted altogether (in which case the system 1209 works autonomously whilst the surgeon performs conventional surgery).

The computerised surgical apparatus 1200 includes a robotic control system 1201 and a tool holder arm apparatus 1210. The tool holder arm apparatus 1210 includes an arm unit 1204 and a surgical device 1208. The arm unit includes an arm (not shown), a control unit 1205, one or more actuators 1206 and one or more force sensors 1207 (e.g. torque sensors). The arm includes one or more joints to allow movement of the arm. The tool holder arm apparatus 1210 sends signals to and receives signals from the robotic control system 1201 via a wired or wireless connection 1211. The robotic control system 1201 includes a control processor 1202 and a database 1203. Although shown as a separate robotic control system, the robotic control system 1201 and the robotic control system 1111 may be one and the same. The surgical device 1208 has the same components as the surgical device 1103. These are not shown in FIG. 6.

In response to control signals received from the robotic control system 1201, the control unit 1205 controls the one or more actuators 1206 to drive the arm about the one or more joints to move it to an appropriate position. The operation of the surgical device 1208 is also controlled by control signals received from the robotic control system 1201. The control signals are generated by the control processor 1202 in response to signals received from one or more of the arm unit 1204, surgical device 1208 and any other signal sources (not shown). The other signal sources may include an imaging device (e.g. imaging device 1102 of the master-slave system 1126) which captures images of the surgical scene. The values of the signals received by the control processor 1202 are compared to signal values stored in the database 1203 along with corresponding arm position and/or surgical device operation state information. The control processor 1202 retrieves from the database 1203 arm position and/or surgical device operation state information associated with the values of the received signals. The control processor 1202 then generates the control signals to be transmitted to the control unit 1205 and surgical device 1208 using the retrieved arm position and/or surgical device operation state information.

For example, if signals received from an imaging device which captures images of the surgical scene indicate a predetermined surgical scenario (e.g. via neural network image classification process or the like), the predetermined surgical scenario is looked up in the database 1203 and arm position information and/or surgical device operation state information associated with the predetermined surgical scenario is retrieved from the database. As another example, if signals indicate a value of resistance measured by the one or more force sensors 1207 about the one or more joints of the arm unit 1204, the value of resistance is looked up in the database 1203 and arm position information and/or surgical device operation state information associated with the value of resistance is retrieved from the database (e.g. to allow the position of the arm to be changed to an alternative position if an increased resistance corresponds to an obstacle in the arm's path). In either case, the control processor 1202 then sends signals to the control unit 1205 to control the one or more actuators 1206 to change the position of the arm to that indicated by the retrieved arm position information and/or signals to the surgical device 1208 to control the surgical device 1208 to enter an operation state indicated by the retrieved operation state information (e.g. turning an electric blade to an “on” state or “off” state if the surgical device 1208 is a cutting tool).

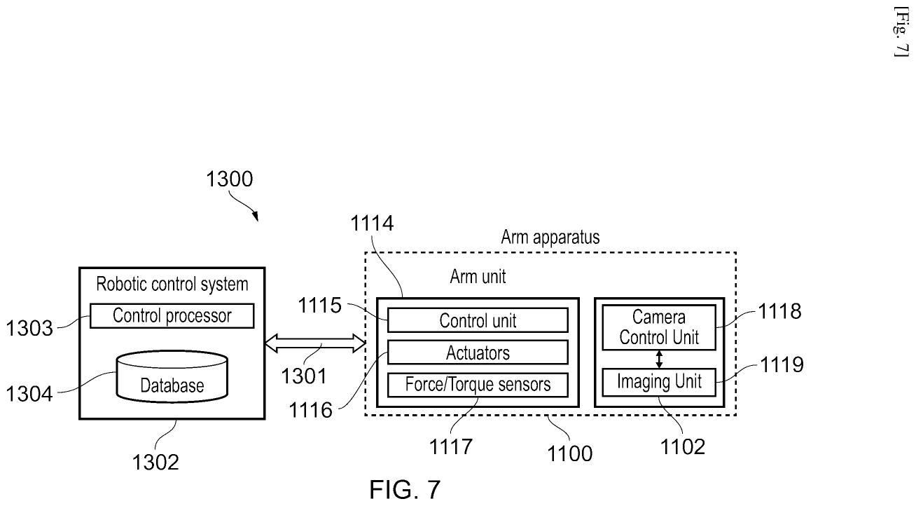

FIG. 7 schematically shows another example of a computer assisted surgery system 1300 to which the present technique is applicable. The computer assisted surgery system 1300 is a computer assisted medical scope system in which an autonomous arm 1100 holds an imaging device 1102 (e.g. a medical scope such as an endoscope, microscope or exoscope). The imaging device of the autonomous arm outputs an image of the surgical scene to an electronic display (not shown) viewable by the surgeon. The autonomous arm autonomously adjusts the view of the imaging device whilst the surgeon performs the surgery to provide the surgeon with an appropriate view of the surgical scene in real time. The autonomous arm 1100 is the same as that of FIG. 5 and is therefore not described. However, in this case, the autonomous arm is provided as part of the standalone computer assisted medical scope system 1300 rather than as part of the master-slave system 1126 of FIG. 5. The autonomous arm 1100 can therefore be used in many different surgical setups including, for example, laparoscopic surgery (in which the medical scope is an endoscope) and open surgery.

The computer assisted medical scope system 1300 also includes a robotic control system 1302 for controlling the autonomous arm 1100. The robotic control system 1302 includes a control processor 1303 and a database 1304. Wired or wireless signals are exchanged between the robotic control system 1302 and autonomous arm 1100 via connection 1301.

In response to control signals received from the robotic control system 1302, the control unit 1115 controls the one or more actuators 1116 to drive the autonomous arm 1100 to move it to an appropriate position for images with an appropriate view to be captured by the imaging device 1102. The control signals are generated by the control processor 1303 in response to signals received from one or more of the arm unit 1114, imaging device 1102 and any other signal sources (not shown). The values of the signals received by the control processor 1303 are compared to signal values stored in the database 1304 along with corresponding arm position information. The control processor 1303 retrieves from the database 1304 arm position information associated with the values of the received signals. The control processor 1303 then generates the control signals to be transmitted to the control unit 1115 using the retrieved arm position information.

For example, if signals received from the imaging device 1102 indicate a predetermined surgical scenario (e.g. via neural network image classification process or the like), the predetermined surgical scenario is looked up in the database 1304 and arm position information associated with the predetermined surgical scenario is retrieved from the database. As another example, if signals indicate a value of resistance measured by the one or more force sensors 1117 of the arm unit 1114, the value of resistance is looked up in the database 1203 and arm position information associated with the value of resistance is retrieved from the database (e.g. to allow the position of the arm to be changed to an alternative position if an increased resistance corresponds to an obstacle in the arm's path). In either case, the control processor 1303 then sends signals to the control unit 1115 to control the one or more actuators 1116 to change the position of the arm to that indicated by the retrieved arm position information.

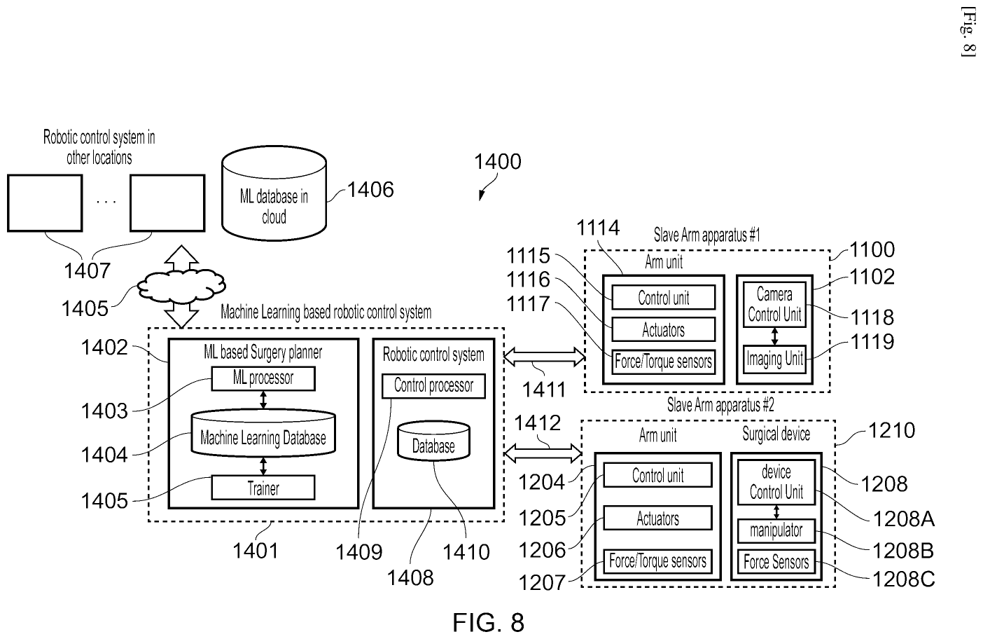

FIG. 8 schematically shows another example of a computer assisted surgery system 1400 to which the present technique is applicable. The system includes one or more autonomous arms 1100 with an imaging unit 1102 and one or more autonomous arms 1210 with a surgical device 1210. The one or more autonomous arms 1100 and one or more autonomous arms 1210 are the same as those previously described. Each of the autonomous arms 1100 and 1210 is controlled by a robotic control system 1408 including a control processor 1409 and database 1410. Wired or wireless signals are transmitted between the robotic control system 1408 and each of the autonomous arms 1100 and 1210 via connections 1411 and 1412, respectively. The robotic control system 1408 performs the functions of the previously described robotic control systems 1111 and/or 1302 for controlling each of the autonomous arms 1100 and performs the functions of the previously described robotic control system 1201 for controlling each of the autonomous arms 1210.

The autonomous arms 1100 and 1210 perform at least a part of the surgery completely autonomously (e.g. when the system 1400 is an open surgery system). The robotic control system 1408 controls the autonomous arms 1100 and 1210 to perform predetermined actions during the surgery based on input information indicative of the current stage of the surgery and/or events happening in the surgery. For example, the input information includes images captured by the image capture device 1102. The input information may also include sounds captured by a microphone (not shown), detection of in-use surgical instruments based on motion sensors comprised with the surgical instruments (not shown) and/or any other suitable input information.

The input information is analysed using a suitable machine learning (ML) algorithm (e.g. a suitable artificial neural network) implemented by machine learning based surgery planning apparatus 1402. The planning apparatus 1402 includes a machine learning processor 1403, a machine learning database 1404 and a trainer 1405.

The machine learning database 1404 includes information indicating classifications of surgical stages (e.g. making an incision, removing an organ or applying stitches) and/or surgical events (e.g. a bleed or a patient parameter falling outside a predetermined range) and input information known in advance to correspond to those classifications (e.g. one or more images captured by the imaging device 1102 during each classified surgical stage and/or surgical event). The machine learning database 1404 is populated during a training phase by providing information indicating each classification and corresponding input information to the trainer 1405. The trainer 1405 then uses this information to train the machine learning algorithm (e.g. by using the information to determine suitable artificial neural network parameters). The machine learning algorithm is implemented by the machine learning processor 1403.

Once trained, previously unseen input information (e.g. newly captured images of a surgical scene) can be classified by the machine learning algorithm to determine a surgical stage and/or surgical event associated with that input information. The machine learning database also includes action information indicating the actions to be undertaken by each of the autonomous arms 1100 and 1210 in response to each surgical stage and/or surgical event stored in the machine learning database (e.g. controlling the autonomous arm 1210 to make the incision at the relevant location for the surgical stage “making an incision” and controlling the autonomous arm 1210 to perform an appropriate cauterisation for the surgical event “bleed”). The machine learning based surgery planner 1402 is therefore able to determine the relevant action to be taken by the autonomous arms 1100 and/or 1210 in response to the surgical stage and/or surgical event classification output by the machine learning algorithm. Information indicating the relevant action is provided to the robotic control system 1408 which, in turn, provides signals to the autonomous arms 1100 and/or 1210 to cause the relevant action to be performed.

The planning apparatus 1402 may be included within a control unit 1401 with the robotic control system 1408, thereby allowing direct electronic communication between the planning apparatus 1402 and robotic control system 1408. Alternatively or in addition, the robotic control system 1408 may receive signals from other devices 1407 over a communications network 1405 (e.g. the internet). This allows the autonomous arms 1100 and 1210 to be remotely controlled based on processing carried out by these other devices 1407. In an example, the devices 1407 are cloud servers with sufficient processing power to quickly implement complex machine learning algorithms, thereby arriving at more reliable surgical stage and/or surgical event classifications. Different machine learning algorithms may be implemented by different respective devices 1407 using the same training data stored in an external (e.g. cloud based) machine learning database 1406 accessible by each of the devices. Each device 1407 therefore does not need its own machine learning database (like machine learning database 1404 of planning apparatus 1402) and the training data can be updated and made available to all devices 1407 centrally. Each of the devices 1407 still includes a trainer (like trainer 1405) and machine learning processor (like machine learning processor 1403) to implement its respective machine learning algorithm.

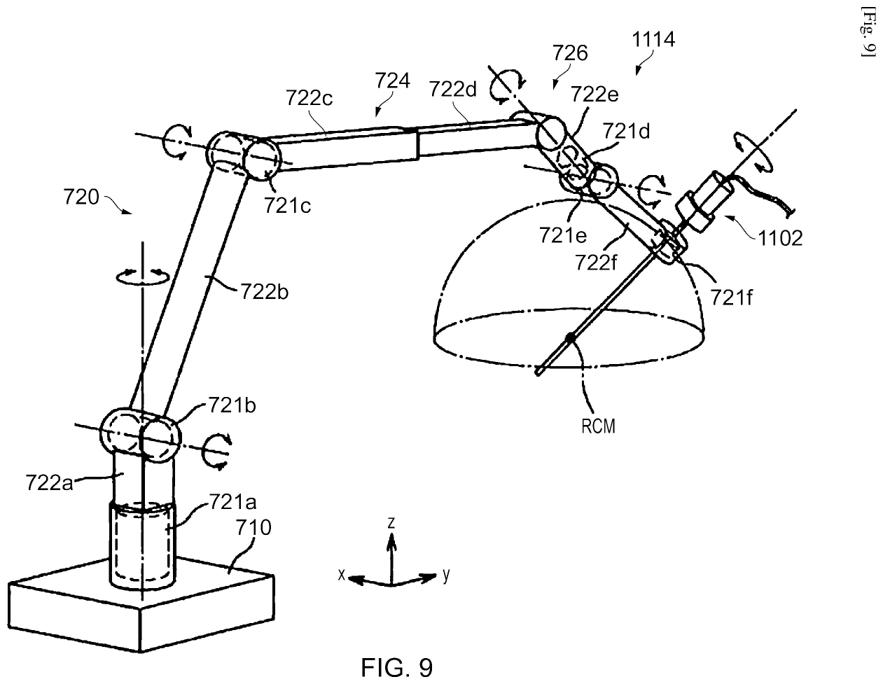

FIG. 9 shows an example of the arm unit 1114. The arm unit 1204 is configured in the same way. In this example, the arm unit 1114 supports an endoscope as an imaging device 1102. However, in another example, a different imaging device 1102 or surgical device 1103 (in the case of arm unit 1114) or 1208 (in the case of arm unit 1204) is supported.

The arm unit 1114 includes a base 710 and an arm 720 extending from the base 720. The arm 720 includes a plurality of active joints 721a to 721f and supports the endoscope 1102 at a distal end of the arm 720. The links 722a to 722f are substantially rod-shaped members. Ends of the plurality of links 722a to 722f are connected to each other by active joints 721a to 721f, a passive slide mechanism 724 and a passive joint 726. The base unit 710 acts as a fulcrum so that an arm shape extends from the base 710.

A position and a posture of the endoscope 1102 are controlled by driving and controlling actuators provided in the active joints 721a to 721f of the arm 720. According to the this example, a distal end of the endoscope 1102 is caused to enter a patient's body cavity, which is a treatment site, and captures an image of the treatment site. However, the endoscope 1102 may instead be another device such as another imaging device or a surgical device. More generally, a device held at the end of the arm 720 is referred to as a distal unit or distal device.

Here, the arm unit 700 is described by defining coordinate axes as illustrated in FIG. 11 as follows. Furthermore, a vertical direction, a longitudinal direction, and a horizontal direction are defined according to the coordinate axes. In other words, a vertical direction with respect to the base 710 installed on the floor surface is defined as a z-axis direction and the vertical direction. Furthermore, a direction orthogonal to the z axis, the direction in which the arm 720 is extended from the base 710 (in other words, a direction in which the endoscope 1102 is positioned with respect to the base 710) is defined as a y-axis direction and the longitudinal direction. Moreover, a direction orthogonal to the y-axis and z-axis is defined as an x-axis direction and the horizontal direction.

The active joints 721a to 721f connect the links to each other to be rotatable. The active joints 721a to 721f have the actuators, and have each rotation mechanism that is driven to rotate about a predetermined rotation axis by drive of the actuator. As the rotational drive of each of the active joints 721a to 721f is controlled, it is possible to control the drive of the arm 720, for example, to extend or contract (fold) the arm unit 720.

The passive slide mechanism 724 is an aspect of a passive form change mechanism, and connects the link 722c and the link 722d to each other to be movable forward and rearward along a predetermined direction. The passive slide mechanism 724 is operated to move forward and rearward by, for example, a user, and a distance between the active joint 721c at one end side of the link 722c and the passive joint 726 is variable. With the configuration, the whole form of the arm unit 720 can be changed.

The passive joint 736 is an aspect of the passive form change mechanism, and connects the link 722d and the link 722e to each other to be rotatable. The passive joint 726 is operated to rotate by, for example, the user, and an angle formed between the link 722d and the link 722e is variable. With the configuration, the whole form of the arm unit 720 can be changed.

In an embodiment, the arm unit 1114 has the six active joints 721a to 721f, and six degrees of freedom are realized regarding the drive of the arm 720. That is, the passive slide mechanism 726 and the passive joint 726 are not objects to be subjected to the drive control while the drive control of the arm unit 1114 is realized by the drive control of the six active joints 721a to 721f.

Specifically, as illustrated in FIG. 11 the active joints 721a, 721d, and 721f are provided so as to have each long axis direction of the connected links 722a and 722e and a capturing direction of the connected endoscope 1102 as a rotational axis direction. The active joints 721b, 721c, and 721e are provided so as to have the x-axis direction, which is a direction in which a connection angle of each of the connected links 722a to 722c, 722e, and 722f and the endoscope 1102 is changed within a y-z plane (a plane defined by the y axis and the z axis), as a rotation axis direction. In this manner, the active joints 721a, 721d, and 721f have a function of performing so-called yawing, and the active joints 421b, 421c, and 421e have a function of performing so-called pitching.

Since the six degrees of freedom are realized with respect to the drive of the arm 720 in the arm unit 1114, the endoscope 1102 can be freely moved within a movable range of the arm 720. FIG. 11 illustrates a hemisphere as an example of the movable range of the endoscope 723. Assuming that a central point RCM (remote center of motion) of the hemisphere is a capturing centre of a treatment site captured by the endoscope 1102, it is possible to capture the treatment site from various angles by moving the endoscope 1102 on a spherical surface of the hemisphere in a state where the capturing centre of the endoscope 1102 is fixed at the centre point of the hemisphere.

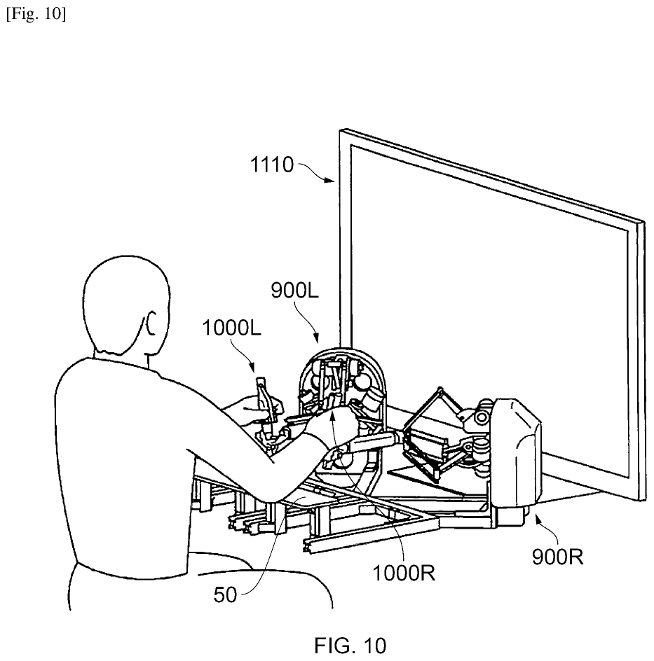

FIG. 10 shows an example of the master console 1104. Two control portions 900R and 900L for a right hand and a left hand are provided. A surgeon puts both arms or both elbows on the supporting base 50, and uses the right hand and the left hand to grasp the operation portions 1000R and 1000L, respectively. In this state, the surgeon operates the operation portions 1000R and 1000L while watching electronic display 1110 showing a surgical site. The surgeon may displace the positions or directions of the respective operation portions 1000R and 1000L to remotely operate the positions or directions of surgical instruments attached to one or more slave apparatuses or use each surgical instrument to perform a grasping operation.

Some embodiments of the present technique are defined by the following numbered clauses:

(1)
A computer assisted surgery system including:
a computerised surgical apparatus; and
a control apparatus;
wherein the control apparatus includes circuitry configured to:
receive information indicating a first region of a surgical scene from which information is obtained by the computerised surgical apparatus to make a decision;
receive information indicating a second region of the surgical scene from which information is obtained by a medical professional to make a decision;
determine if there is a discrepancy between the first and second regions of the surgical scene; and
if there is a discrepancy between the first and second regions of the surgical scene:
perform a predetermined process based on the discrepancy.

(2)
A computer assisted surgery system according to clause 1, wherein:
the first region is a region from which information was obtained by the computerised surgical apparatus when a computer decision was made by the computerised surgical apparatus; and
the control apparatus includes circuitry configured to:
receive information indicating the computer decision of the computerised surgical apparatus;
as the predetermined process, control the computerised surgical apparatus to make an updated computer decision based on the discrepancy.

(3)
A computer assisted surgery system according to clause 2, wherein the updated computer decision is based on the second region.

(4)
A computer assisted surgery system according to clauses 2 or 3, the control apparatus including circuitry configured to control the computerised surgical apparatus to:
adjust one or more parameters used by the computerised surgical apparatus to determine the first region of the surgical scene; and
make the updated computer decision using the adjusted one or more parameters.

(5)
A computer assisted surgery system according to any one of clauses 2 to 4, the control apparatus including circuitry configured to:
receive information indicating the updated computer decision of the computerised surgical apparatus;
output information indicating the updated computer decision to the medical professional;
receive information indicating if the medical professional approves the updated computer decision;
if the medical professional approves the updated computer decision, control the computerised surgical apparatus to perform an action based on the updated computer decision.

(6)
A computer assisted surgery system according to any preceding clause, the control apparatus including circuitry configured to output information indicating the first and second regions of the surgical scene to the medical professional.

(7)
A computer assisted surgery system according to clause 6:
wherein the information indicating the first region of the surgical scene includes an image of the surgical scene and information indicating the first region in the image; and
wherein the information indicating the second region of the surgical scene includes an image of the surgical scene and information indicating the second region of the surgical scene in the image.

(8)
A computer assisted surgery system according to any preceding clause, wherein the information indicating the first region of the surgical scene includes an artificial intelligence attention heat map.

(9)
A computer assisted surgery system according to any preceding clause, wherein the information indicating the second region of the surgical scene includes gaze tracking information of the medical professional.

(10)
A computer assisted surgery system according to clause 4, wherein the adjustable one or more parameters used by the computerised surgical apparatus to determine the first region of the surgical scene include one or more of a light parameter of the surgical scene, a viewing angle of the surgical scene, a pixel value of an image of the surgical scene used by the computerised surgical apparatus to determine the first region of the surgical scene and an image type of an image of the surgical scene used by the computerised surgical apparatus to determine the first region of the surgical scene.

(11)
A computer assisted surgery system according to clause 5, wherein:
the updated computer decision is based on images of the surgical scene captured using a plurality of imaging modalities; and
the control apparatus includes circuitry configured to output the images of the surgical scene captured using the plurality of imaging modalities to the medical professional with the information indicating the updated computer decision.

(12)
A computer assisted surgery system according to clause 1, wherein:
the computerised surgical apparatus obtains information from the first region and the medical professional obtains information from the second region via a captured image of the surgical scene, the captured image of the surgical scene including a cropped portion visible to the computerised surgical apparatus but not visible to the medical professional; and
if the first region is within the cropped portion, the predetermined process includes at least one of:
controlling the computerised surgical apparatus to adjust a view of a camera used to capture the image of the surgical scene so the first region is in a non-cropped region of the image;
outputting information indicating the need to adjust a view of a camera used to capture the image of the surgical scene so the first region is in a non-cropped region of the image to the medical professional.

(13)
A computer assisted surgery system according to any preceding clause, wherein:
the computerised surgical apparatus includes circuitry configured to determine an operating parameter of a surgical tool of the computerised surgical apparatus and if a value of the operating parameter falls outside a predetermined range; and
the control apparatus includes circuitry configured to:
if the value of the operating parameter of the surgical tool falls outside the predetermined range, determine if the surgical tool is within the second region; and
if the surgical tool is not within the second region;
output information indicating that the value of the operating parameter of the surgical tool falls outside the predetermined range to the medical professional.

(14)
A computer assisted surgery system according to any preceding clause, wherein the computer assisted surgery system is a computer assisted medical vision scope system, a master-slave system or an open surgery system.

(15)
A surgical control apparatus including circuitry configured to:
receive information indicating a first region of a surgical scene from which information is obtained by a computerised surgical apparatus to make a decision;
receive information indicating a second region of the surgical scene from which information is obtained by a medical professional to make a decision;
determine if there is a discrepancy between the first and second regions of the surgical scene; and
if there is a discrepancy between the first and second regions of the surgical scene:
perform a predetermined process based on the discrepancy.

(16)
A surgical control method including:
receive information indicating a first region of a surgical scene from which information is obtained by a computerised surgical apparatus to make a decision;
receive information indicating a second region of the surgical scene from which information is obtained by a medical professional to make a decision;
determine if there is a discrepancy between the first and second regions of the surgical scene; and
if there is a discrepancy between the first and second regions of the surgical scene:
perform a predetermined process based on the discrepancy.

(17)
A program for controlling a computer to perform a surgical control method according to clause 16.

(18)
A non-transitory storage medium storing a computer program according to clause 17.

Numerous modifications and variations of the present disclosure are possible in light of the above teachings. It is therefore to be understood that within the scope of the appended claims, the disclosure may be practiced otherwise than as specifically described herein.

In so far as embodiments of the disclosure have been described as being implemented, at least in part, by software-controlled data processing apparatus, it will be appreciated that a non-transitory machine-readable medium carrying such software, such as an optical disk, a magnetic disk, semiconductor memory or the like, is also considered to represent an embodiment of the present disclosure.

It will be appreciated that the above description for clarity has described embodiments with reference to different functional units, circuitry and/or processors. However, it will be apparent that any suitable distribution of functionality between different functional units, circuitry and/or processors may be used without detracting from the embodiments.

Described embodiments may be implemented in any suitable form including hardware, software, firmware or any combination of these. Described embodiments may optionally be implemented at least partly as computer software running on one or more data processors and/or digital signal processors. The elements and components of any embodiment may be physically, functionally and logically implemented in any suitable way. Indeed the functionality may be implemented in a single unit, in a plurality of units or as part of other functional units. As such, the disclosed embodiments may be implemented in a single unit or may be physically and functionally distributed between different units, circuitry and/or processors.

Although the present disclosure has been described in connection with some embodiments, it is not intended to be limited to the specific form set forth herein. Additionally, although a feature may appear to be described in connection with particular embodiments, one skilled in the art would recognize that various features of the described embodiments may be combined in any manner suitable to implement the technique.

### CITATION LIST

### Non Patent Literature

NPL 1: Jinkyu Kim and John Canny, 2017, 2017 IEEE International Conference on Computer Vision (ICCV), http://openaccess.thecvf.com/content_ICCV_2017/papers/Kim_Interpretable_Learning_for_ICCV_2017_paper.pdf

1. A computer assisted surgery system comprising:
a computerised surgical apparatus; and
a control apparatus;
wherein the control apparatus comprises circuitry configured to:
receive information indicating a first region of a surgical scene from which information is obtained by the computerised surgical apparatus to make a decision;
receive information indicating a second region of the surgical scene from which information is obtained by a medical professional to make a decision;
determine if there is a discrepancy between the first and second regions of the surgical scene; and
if there is a discrepancy between the first and second regions of the surgical scene:
perform a predetermined process based on the discrepancy.

2. A computer assisted surgery system according to claim 1, wherein:
the first region is a region from which information was obtained by the computerised surgical apparatus when a computer decision was made by the computerised surgical apparatus; and
the control apparatus comprises circuitry configured to:
receive information indicating the computer decision of the computerised surgical apparatus;
as the predetermined process, control the computerised surgical apparatus to make an updated computer decision based on the discrepancy.

3. A computer assisted surgery system according to claim 2, wherein the updated computer decision is based on the second region.

4. A computer assisted surgery system according to claim 2, the control apparatus comprising circuitry configured to control the computerised surgical apparatus to:
adjust one or more parameters used by the computerised surgical apparatus to determine the first region of the surgical scene; and
make the updated computer decision using the adjusted one or more parameters.

5. A computer assisted surgery system according to claim 2, the control apparatus comprising circuitry configured to:
receive information indicating the updated computer decision of the computerised surgical apparatus;
output information indicating the updated computer decision to the medical professional;
receive information indicating if the medical professional approves the updated computer decision;
if the medical professional approves the updated computer decision, control the computerised surgical apparatus to perform an action based on the updated computer decision.

6. A computer assisted surgery system according to claim 1, the control apparatus comprising circuitry configured to output information indicating the first and second regions of the surgical scene to the medical professional.

7. A computer assisted surgery system according to claim 6:
wherein the information indicating the first region of the surgical scene comprises an image of the surgical scene and information indicating the first region in the image; and
wherein the information indicating the second region of the surgical scene comprises an image of the surgical scene and information indicating the second region of the surgical scene in the image.

8. A computer assisted surgery system according to claim 1, wherein the information indicating the first region of the surgical scene comprises an artificial intelligence attention heat map.

9. A computer assisted surgery system according to claim 1, wherein the information indicating the second region of the surgical scene comprises gaze tracking information of the medical professional.

10. A computer assisted surgery system according to claim 4, wherein the adjustable one or more parameters used by the computerised surgical apparatus to determine the first region of the surgical scene comprise one or more of a light parameter of the surgical scene, a viewing angle of the surgical scene, a pixel value of an image of the surgical scene used by the computerised surgical apparatus to determine the first region of the surgical scene and an image type of an image of the surgical scene used by the computerised surgical apparatus to determine the first region of the surgical scene.

11. A computer assisted surgery system according to claim 5, wherein:
the updated computer decision is based on images of the surgical scene captured using a plurality of imaging modalities; and
the control apparatus comprises circuitry configured to output the images of the surgical scene captured using the plurality of imaging modalities to the medical professional with the information indicating the updated computer decision.

12. A computer assisted surgery system according to claim 1, wherein:
the computerised surgical apparatus obtains information from the first region and the medical professional obtains information from the second region via a captured image of the surgical scene, the captured image of the surgical scene comprising a cropped portion visible to the computerised surgical apparatus but not visible to the medical professional; and
if the first region is within the cropped portion, the predetermined process comprises at least one of:
controlling the computerised surgical apparatus to adjust a view of a camera used to capture the image of the surgical scene so the first region is in a non-cropped region of the image;
outputting information indicating the need to adjust a view of a camera used to capture the image of the surgical scene so the first region is in a non-cropped region of the image to the medical professional.

13. A computer assisted surgery system according to claim 1, wherein:
the computerised surgical apparatus comprises circuitry configured to determine an operating parameter of a surgical tool of the computerised surgical apparatus and if a value of the operating parameter falls outside a predetermined range; and
the control apparatus comprises circuitry configured to:
if the value of the operating parameter of the surgical tool falls outside the predetermined range, determine if the surgical tool is within the second region; and
if the surgical tool is not within the second region;
output information indicating that the value of the operating parameter of the surgical tool falls outside the predetermined range to the medical professional.

14. A computer assisted surgery system according to claim 1, wherein the computer assisted surgery system is a computer assisted medical vision scope system, a master-slave system or an open surgery system.

15. A surgical control apparatus comprising circuitry configured to:
receive information indicating a first region of a surgical scene from which information is obtained by a computerised surgical apparatus to make a decision;
receive information indicating a second region of the surgical scene from which information is obtained by a medical professional to make a decision;
determine if there is a discrepancy between the first and second regions of the surgical scene; and
if there is a discrepancy between the first and second regions of the surgical scene:
perform a predetermined process based on the discrepancy.

16. A surgical control method comprising:
receive information indicating a first region of a surgical scene from which information is obtained by a computerised surgical apparatus to make a decision;
receive information indicating a second region of the surgical scene from which information is obtained by a medical professional to make a decision;
determine if there is a discrepancy between the first and second regions of the surgical scene; and
if there is a discrepancy between the first and second regions of the surgical scene:
perform a predetermined process based on the discrepancy.

17. A program for controlling a computer to perform a surgical control method according to claim 16.

18. A non-transitory storage medium storing a computer program according to claim 17.
